(19) United States
(12) Patent Application Publication
(10) Pub. No.: US 2017/0233760 A1
(43) Pub. Date: Aug. 17, 2017

(54) BIOLOGICALLY ACTIVE NUCLEOTIDE MOLECULES FOR SELECTIVELY KILLING OFF CELLS, USE THEREOF, AND APPLICATION KIT

(71) Applicants: Tobias POEHLMANN, Zwickau (DE); Rolf GUENTHER, Hamburg (DE)

(72) Inventors: Tobias POEHLMANN, Zwickau (DE); Rolf GUENTHER, Hamburg (DE)

(73) Assignee: FRIEDRICH-SCHILLER-UNIVERSITAET JENA, Jena (DE)

(21) Appl. No.: 13/979,084

(22) PCT Filed: Jan. 20, 2012
(86) PCT No.: PCT/EP12/50879

§ 371 Date: Sep. 26, 2013

(30) Foreign Application Priority Data
Jan. 21, 2011 (DE) 10-2011-009-470.9

(51) Int. Cl. C12N 15/85; C12N 15/113

(52) U.S. Cl. CPC C12N 15/85; C12N 15/1137; C12N 15/1138; C12N 2310/14; C12N 2310/11; C12N 2310/3181; C12N 2310/3231

(57) ABSTRACT

Biologically active nucleotide molecules are configured, with the nucleotide sequence thereof, to be able to trigger several, in particular a plurality of “off-target” effects to cause cell-killing stress by means of binding of same, by means of which off-target effects cells are so massively influenced that the cells die off or programmed cell death (apoptosis) is induced in the cells.

The invention relates to biologically active molecules on the basis of nucleotides which allow the selective killing of cells, to the use of said biologically active molecules and to an application kit for use thereof.

Methods which are to selectively kill biological cells conventionally use physical means such as UV radiation, heat (Hsie A W, Brimer P A, Mitchell T J, Gosslee D G. The dose-response relationship for ultraviolet-light-induced mutations at the hypoxanthine-guanine phosphoribosylthransferase locus in Chinese hamster ovary cells. Somatic Cell Genet. 1975 October; 1(4):383-9; Gillespie E H, Gibbons S A. Autoclaves and their dangers and safety in laboratories. J. Hyg (Lond). 1975 December; 75(3):475-87.) or chemical substances, for example acids, bases, formaldehydes (National Toxicology Program. Final Report on Carcinogens Background Document for Formaldehyde. Rep Carcinog Backgr Doc. 2010 January; (10-5981):i-512.) which destroy the structure of the cell as such. These agents are often harmful to the environment and can hardly be used in the organism. In order to kill cells in an organism, biochemicial agents (protein inhibitors, antagonists, cytostatics etc.) are used (Tanaka S, Arii S. Current status of molecularly targeted therapy for heptacellular carcinoma: basic science. Int. J Clin Oncol. 2010 June; 15(3):235-41. Epub 2010 May 27.), which have a strong effect on the physiology of cells and, thus, may also lead to the death of the cell. However, with none of these methods, it is possible to selectively kill specific types of cells since these substances have the same effect on all cells.

A molecular biological approach to selectively act on cells consists in the use of short double-stranded RNA. These so-called siRNA (short interfering RNA) molecules can interact with the mRNA of the target gene in a classical way after their activation and, together with specific endoribonucleases, they form an RNA-protein complex referred to as “RISC” (RNA induced silencing complex). The RISC complex binds to the target mRNA with endonucleases cleaving the target mRNA. In this way, gene expression is prevented and, thus, the generation of target proteins is inhibited.

The inhibition of gene expression by the introduction of short (19-23 bp) double-stranded RNA molecules (siRNA) in eukaryotic cells which are specific for a sequence segment of the mRNA of the target gene has already been described (Elbashir S M et al.: Duplexes of 21-nucleotide RNAs mediate RNA interference in cultured mammalian cells, Nature, 2001, May 24, 411(6836), 494-8; Liu Y et al.: Efficient and isoform-selective inhibition of cellular gene expression by peptide nucleic acids, Biochemistry, 2004 Feb. 24, 43(7), 1921-7; U.S. Pat. No. 5,898,031 A; U.S. Pat. No. 7,056,704 B2).

The use of such molecules does not prevent the transcription of a gene and the production of an mRNA, but the siRNA initiates a cell mechanism which degrades the target mRNA. Finally, as described above, the generation of a specific protein is suppressed without interfering with the expression of further genes (post-transcriptional gene silencing).

At present, the use of siRNA often aims at suppressing exclusively the expression of one single gene in a cell. Thus, effects which silence several genes at the same time or in an unspecific manner are undesirable and, for this reason, the sequences of the mRNA are designed in such a way that these effects are suppressed.

Methods aiming at increasingly transfecting cells of a target tissue in vivo with siRNA (Ikeda et al.: “Ligand-Targeted Delivery of Therapeutic siRNA”, Pharmaceutical Research, Vol. 23, No. 8, August 2006) or at achieving cell specificity by the binding of short peptides which are cleaved in a cell-specific manner (WO 2008/098569 A2) were also developed. By using said modified siRNA molecules, it is possible to selectively reduce or suppress the expression of genes in specific cells.

If the siRNA sequence used is specific for genes that are crucial for the survival of the cell, this may result in the death of the cell. Based on the mechanisms mentioned, this process can, if necessary, also be applied to specific cells.

However, it is a disadvantage that the knock-out of one single gene or of a few genes often does not necessarily lead to the death of the relevant cell; in many cases, it would be necessary to specifically knock-out several genes at the same time in order to produce the desired effect. With respect to therapeutic uses, for example, it would be desirable if cells could be killed directly by siRNA molecules. This would allow to very specifically kill tumor cells or virus infected cells, in particular by using the methods mentioned.

Furthermore, a common problem is that, in the genome of tumour cells or virus-infected cells, mutations are frequent and that, for this reason, the siRNa molecules used may no longer be active and that, thus, the intended interference with the cells fails or, at least, cannot be used in an efficient manner.

The problem underlying the invention is to kill cells, in a wide application area, efficiently, reliably and as effectively as possible in the organism without the aforementioned disadvantages of chemical, physical, biochemical or molecular biological methods known per se.

According to the invention, the biologically active nucleotide molecules for example based on RNA, siRNA, PNA or LNA, with their nucleotide sequence, which allows binding to the mRNA of several genes, are directed to trigger several, in particular a plurality of “off-target” effects for cell-killing stress situations by binding to said genes.

The term “biologically active nucleotide molecules” encompasses nucleotide molecules of the invention which are functional under all the conditions and in all the applications described herein. The biologically active nucleotide molecules show their action, in particular, by triggering so-called “off-target” effects. According to the invention, “off-target” effects which trigger cell-killing stress situations are to be understood as biological activities and processes in which one nucleotide sequence has several target mRNA sequences and potentially has an effect on the expression of several genes or triggers cell stress independently of the effect on the expression of genes.

Said stress situation, independently of the classic use of the nucleotide molecules, in particular of siRNA, for the reduction of the expression of an individual gene, has such strong effect on the cell via the unspecific nucleotide sequence that the cell dies off or programmed death (apoptosis) is initiated in the cell.

As described at the beginning, nucleotide molecules, for example on the basis of siRNA, having a nucleotide sequence designed for mRNA binding are well known per se, however, in these cases, the nucleotide sequence is designed specifically for the mRNA of one or a few genes in order to achieve a well defined gene manipulation in the cell and, thus, a gene-specific effect on the cell by selectively binding to the target gene.

The nucleotide sequence of the invention is purposefully designed in such a way that it can dock to several mRNAs, in particular a plurality of mRNAs, optionally irrespective of whether the potential mRNAs of the genes that are suitable for binding are actually present in the cell or not. Thus, the primary aim of the intended mRNA binding according to the invention is not gene manipulation directed to cell activity as mentioned above, but the aim is to trigger as many “off-target” effects as possible, which, up to the present, if possible, were to be avoided or to be reduced with respect to targeted action on genes, using in particular a plurality of (in principle arbitrary) mRNA bindings of the nucleotide molecules. Rather, the “off-target” effects, as many of them as possible, are used to cause an extreme stress situation for the cell which the target cell cannot cope with and by means of which said target cell is purposefully killed (not by targeted manipulation of gene expression but by general stress).

Manipulation of specific genes which is known to necessarily occur in the context of gene binding and which has an impact on cell activity is a secondary effect and, depending on the effect of the gene manipulation, might possibly further facilitate the action on the cell (in addition to the stress situation intended by the invention).

Thus, the selection of the target genes which can be bound by the nucleotide sequence is not, or at least not primarily, the effect to be achieved by an intended gene manipulation which is to have an effect on cells, but it is determined by the intended effect of the “off-target” effects which can be achieved by means of gene bindings and the stress situation which is triggered with these in the cell.

In order to produce said “off-target” effects, the nucleotide sequences are selected in such a way that they do not correspond to one target gene, as is usual, but that they correspond to as many target genes of the cells as possible. Thus, a nucleotide interference which has a toxic effect on a plurality of genes is created and the physiology of the cell is affected drastically.

The suggested use can be applied in combination with known mechanisms for achieving cell specificity and with known means for the stabilization e.g. of siRNA and for enhanced introduction of the nucleotide molecules into cells.

The nucleotide sequences suggested are not restricted to the use as classic siRNA; also short (10-20 bp) double-stranded or single-stranded RNA, long (20-300 bp) double- or single-stranded RNA; DNA or chemical analogs, such as PNA, can be used with the nucleotide sequences suggested.

In addition to the induction of the “off-target” effects, known stress-inducing nucleotide sequence orders can support the cell-damaging effect of the biologically active nucleotide molecules (FEDOROV Y et al., Off-target effects by siRNA can induce toxic phenotype. RNA (2006), 12:1188-1196).

By means of a suitable transfection system, such as e.g. nanoparticles polyethylenimine or liposomes, the active ingredient molecules can be introduced into the cells according to known methods.

Molecule constructs can further be bound to other substances (e.g. to nanoparticles as carrier system or to fluorochromes) for improved transport into or to the cells as well as for their stabilization or their detection.

The biologically active nucleotide molecules are suited for the selective killing of eukaryotic cells, in particular animal, plant or fungal cells as well as virus-infected and prokaryotic cells.

If biologically active nucleotide molecules are used, they may also be used in combination with protease inhibitors.

An application kit for use and administration of the biologically active nucleotide molecules, consisting of at least
at least one ampoule (ampoule A) which contains the biologically active molecule and may further contain:
at least one further ampoule (ampoule B) with a transfection system, for example, nanoparticles, polyethyleneimines or lipids,
at least one further ampoule (ampoule C) which contains further components for binding to the biologically active molecules or the transfection system,
dilution and reaction buffers for the contents of ampoules A, B and C,
one or more probes and syringes with cannulas and other required materials for injecting the mixture from the ampoule contents into the medium containing the target cells as well as
instructions for use and administration
   is advantageous.

In the following, the invention is exemplified in detail by embodiments illustrated in the Figures.

The Figures show:

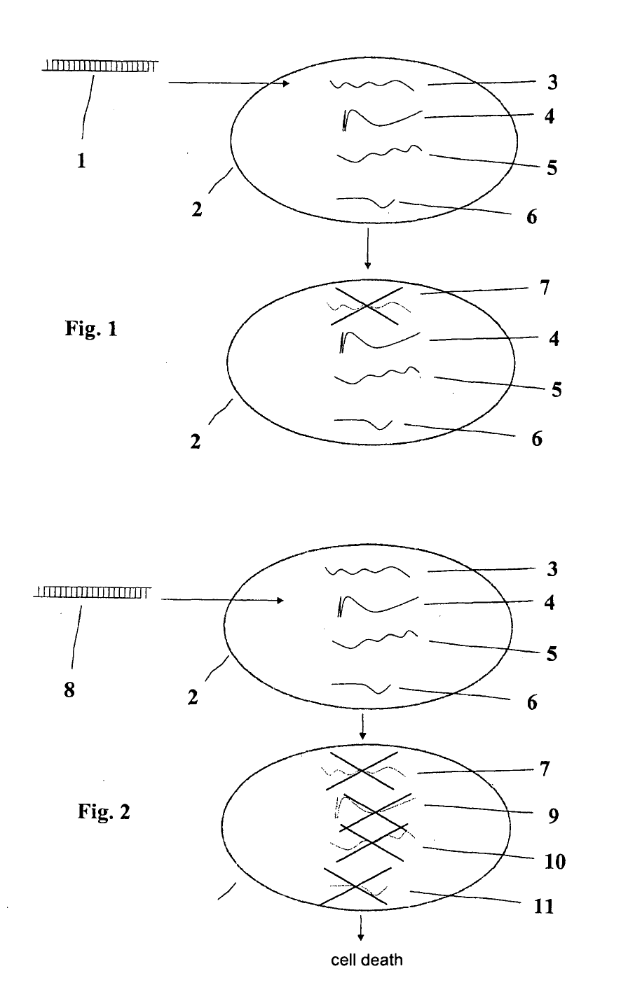

FIG. 1: Schematic diagram of a known siRNA which is introduced into a cell, is specific for an mRNA and suppresses the expression of a target gene.

FIG. 2: Schematic diagram of a siRNA of the invention which is introduced into a cell and triggers as many unspecific RNAi effects (off-target effects) as possible in the cell.

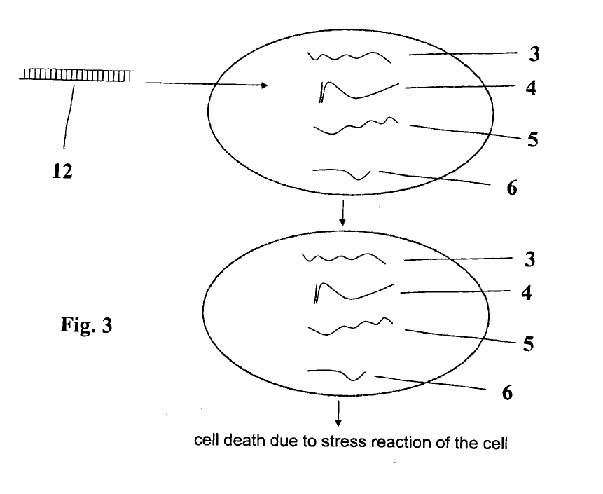

FIG. 3: Schematic diagram of a siRNA which is introduced into a cell and is not to reduce the expression of genes and the degradation of mRNAs in the cell but is to cause cell death by means of stress reactions in the cell which are triggered by specific sequence segments of the siRNA.

FIG. 1 shows the mechanism of a conventional and known siRNA 1 which is introduced into a cell 2 (as illustrated by the arrow) and has a specific nucleotide sequence (not explicitly shown) for binding to a first gene-specific mRNA 3 nucleotide sequence. Subsequently, the siRNA 1 is introduced into the RNA-induced silencing complex (RISC) (not explicitly shown either) which separates the siRNA 1 in its two single strands and the antisense strand of the siRNA 1 together with the RISC anneals to the first mRNA 3. Then, the gene-specific first mRNA 3 is cleaved and fragmented whereby the expression of a target gene, based on the first mRNA 3, is suppressed (cf. degraded first mRNA 7 in FIG. 1). The siRNA which is thus set free and integrated into the RISC anneals to the next specific first mRNA 3 present in the cell 2 and causes its degradation. The aim is that each siRNA 1 binds to one specific first mRNA 3 only and causes its degradation. In contrast to the first mRNA 3, any second further mRNA 4, third further mRNA 5 and fourth further mRNA 6 present in the cell 2 are not affected by the siRNA 1 and its nucleotide sequence (not explicitly shown), respectively, so that the expression of the genes corresponding to mRNA 4-6 is not modified. This method is well known.

FIG. 2 shows, for comparison, the mechanism of a siRNA 8 of the invention which is introduced into the cell 2 (also illustrated by an arrow) which, in an exemplary manner, contains the first mRNA 3, the second mRNA 4, the third mRNA 5 and the fourth mRNA 6.

The suggested siRNA 8 contains a chain of one or more of the following nucleotide sequences (not explicitly illustrated for the sake of clarity)

GGUA, CGUC, CGUU, CCAA,
AAGG, GGUG, CUCG, CUCC, CUCU, CUUA, GGUC, GGUU,
AAAG, AAAC, AAAU, AAGA, AAGC, AAGU, AACA, AACG,
AACC, AACU, AAUA, CUUU, AAUG, AAUC, AAUU, AGGA,
AGUG, AGUC, AGUU, ACAA, ACAG, ACAC, ACAU, ACGA,
ACGG, ACGC, ACGU, ACCA, CAUU, CGAA, ACCG, ACCC,
ACCU, ACUA, ACUG, ACUC, ACUU, AUAA, GGAG, GGAC,
GGAU, GGGA, GGGC, GGGU, GGCA, GGCG, GGCC, GGCU,
GCAA, GCAG, GCAC, GCAU, AUAG, AUAC, AUAU, AUGA,
AUGG, AUGC, AUGU, AUCA, CGCG, CGCC, CGCU, AUCG,
AUCC, AUCU, AUUA, AUUG, AUUC, AUUU, GAAA, GAAG,
GAAC, GAAU, GAGA, GAGG, GAGC, GAGU, GACA, GACG,
GACC, GACU, GAUA, GAUG, GAUC, GAUU, GGAA, GCGA,
GCGG, GCGC, GCGU, GCCA, GCCG, GCCC, GCCU, GCUA,
GCUG, GCUC, GCUU, GUAA, GUAG, GUAC, GUAU, GUGA,
GUGG, GUGC, GUGU, GUCA, GUCG, GUCC, GUCU, GUUA,
GUUG, GUUC, GUUU, CAAA, CAAG, CAAC, CAAU, CAGA,
CAGG, CAGC, CAGU, CACA, CACG, CACC, CACU, CAUA,
CAUG, CAUC, CGAG, CGAC, CGAU, CGGA, CGGG, CGGC,
CGGU, CGCA, CGUA, CGUG, CCAG, CCAC, CCAU, CCGA,
CCGG, CCGC, CCGU, CCCA, AGAA, AGAG, AGAC, AGAU,
CCCG, CCCU, AGGG, AGGC, AGGU, AGCA, CCUA, CCUG,
CCUC, CCUU, CUAA, CUAG, CUAC, CUAU, AGCG, AGUA,
CUGA, CUGG, CUGC, CUGU, CUCA, CUUG, CUUC, AGCC,
AGCU

so that, in contrast to FIG. 1, the nucleotide sequences, which are bound in the chain, as a whole do not have a degrading effect on only one of the mRNAs 3-6 but on several or a plurality of the mRNA molecules, and thus bind all the mRNA set forth in FIG. 2 (mRNA 3-6). It is possible that at least one selected nucleotide sequence binds to several or all of the shown mRNA molecules (mRNA 3-6) or that one of the selected nucleotide sequences selectively acts on one specific mRNA 3-6. It is crucial that as many of the mRNA 3-6 as possible (preferably all) are bound and degraded by the chain (total number of all nucleotide sequences) of siRNA 8 (cf. degraded first to fourth mRNA 7, 9, 10, 11 in FIG. 2).

Due to the degradation of said plurality of mRNA molecules (in a simplifying manner, the present Example shows only four mRNA molecules), several to numerous unspecific RNAi effects (off-target effects) are triggered in that the siRNA 8 suppresses the expression of several to numerous genes (cf. degraded mRNA 7, 9, 10 in FIG. 2) by means of (ideally) only one nucleotide sequence with the aim to kill the cell 2 in this way, wherein the cell dies due to the massive effect of the siRNA 8.

For example, by means of the siRNA 8 having the nucleotide sequence (5′-3′) UUAACUGUAUCUGGAGCtt (SEQ ID NO:3), it is possible to degrade the mRNA of the genes suppressor of cytokine signaling-1 (SOCS1, NM_003745.1), N-acetylneuraminic acid phosphatase (NANP, NM_152667.2) transmembrane protein 215 (TMEM215, NM_212558.2) and of the CD81 molecule (CD81, NM_004356.3).

A nucleotide sequence AACUGUAUCUGGAGCtt (SEQ ID NO:4) of the siRNA 8 is specifically active for the mRNAs of the genes suppressor of cytokine signaling-1 (SOCS1, NM_003745.1) and N-acetylneuraminic acid phosphatase (NANP, NM_152667.2). A nucleotide sequence GGCUGAACAAAGGAGAtt (SEQ ID NO:6) acts specifically on the major histocompatibility complex, class-I, G (HLA-G, NM_002127.4), glycerol kinase 5 (putative) (GK5, NM_001039547.1) and DIP2 disco-interacting protein 2 homolog C (NM_014974.2).

In analogy, the siRNA 8 with the sequence GCUCACCAAUGGAGAtt (SEQ ID NO:5) acts specifically on the complement component (3b/4b) receptor 1 (Knops blood group) (CR1, NM-000651.4), transcript variant S, complement component (3b/4b) receptor 1 (Knops blood group) (CR1, NM_000573.3), transcript variant F and glutathione S-transferase alpha 4 (GSTA4, NM_001512.3).

As further examples for the nucleotide sequence of siRNA 8 sequence UGGCUGGCUGGCUGGCtt (SEQ ID NO:7) advantageous against pyroglutamyl peptidase I (PGPEP1, NM_017712.2), rap guanine nucleotide exchange factor (GEF) 3 (RAPGEF3, NM_006105.5), transcript variant 2 and against the retinoid X receptor, alpha (RXRA, NM_002957.4) and sequence GUCUAUCAGCACAAUtt (SEQ ID NO:1) against the signal transducer and activator of transcription 3 (acute-phase response factor) (STAT3, NM_213662.1), transcript variant 3, signal transducer and activator of transcription 3 (acute-phase response factor) (STAT3, NM 003150.3), transcript variant 2, the signal transducer and activator of transcription 3 (acute-phase response factor) (STAT3, NM_139276.2), transcript variant 1, protocadherin alpha 9 (PCDHA9, NM_014005.3) and secernin 3 (SCRN3, NM_024583.3) are mentioned.

As an alternative to the above-mentioned examples of nucleotide sequences of siRNA 8 which are directed against concrete genes, it is also possible to use a nucleotide sequence which has no homology to a human mRNA and, thus, has no direct target gene. In this case, sequences which are known in the state of the art to trigger cell stress can be used. Such nucleotide sequence can have the sequence GCUUAACUGUAUCUGGAGCtt (SEQ ID NO:2).

As can be taken from the nucleotide sequences listed above, modified nucleotides are added to the 3′ end of said sequences, with “t” being 2′-deoxythymidine according to the invention. Into the nucleotide sequences shown above, two 2′-deoxynucleotides are added at the 3′ end and these terminal nucleotides are designated “tt”. However, the structure of these overhangs is not limited to the “tt” overhangs mentioned herein since the type of overhangs per se is not crucial for the effect of the siRNAs as described herein according to the invention. It is also possible to use other overhangs known to the person skilled in the art.

The biologically active nucleotide molecules of the invention can also be used as a pharmaceutical composition. It is for example possible to directly kill cells using the siRNA molecules of the invention for therapeutic applications. Thus, it is possible to selectively kill specific tumour cells or virus-infected cells. For this reason, the nucleotide sequences suggested, i.e. the biologically active nucleotide molecules described above, can be used in the treatment and/or prophylaxis of tumour diseases or virus-induced diseases. Virus-induced diseases within the meaning of the invention comprise diseases which are, for example, caused by herpes viruses, papillomaviruses or HIV viruses. Thus, the virus-induced diseases comprise diseases such as hepatitis, cervical cancer or AIDS.

The present invention also comprises the biologically active nucleotide molecules of the invention for use in the treatment and/or prophylaxis of tumour diseases. Tumour diseases which are treated with the pharmaceutical composition of the invention comprise mamma carcinomas, ovary carcinomas, bronchial carcinomas, colon carcinomas, melanomas, urinary bladder carcinomas, gastric carcinomas, head and neck carcinomas, brain tumours, cervical tumours, prostate carcinomas, testicular carcinomas, bone tumours, renal carcinomas, pancreatic carcinomas, esophageal carcinomas, malignant lymphomas, non-Hodgkin lymphomas, Hodgkin lymphomas and thyroid lymphomas.

In a further preferred embodiment, the biologically active nucleotide molecules, nucleotides or nucleotide analogs can optionally be used in combination with protease inhibitors, as already mentioned above. Corresponding protease inhibitors are known to the skilled person from the state of the art. Inhibitors of hepatitis C protease or inhibitors of HIV protease are mentioned as examples of the protease inhibitors while the present invention is not limited to these.

Moreover, in another preferred embodiment of the invention, the biologically active nucleotide molecules, nucleotides or nucleotide analogs of the invention can optionally be formulated in combination with a “pharmacologically acceptable” carrier and/or solvent. Examples of particularly suited pharmacologically acceptable carriers are known to the person skilled in the art and comprise buffered saline, water, emulsions such as e.g. oil/water emulsions, different types of detergents, sterile solutions etc.

Pharmaceutical compositions within the meaning of the invention comprising the pharmacologically acceptable carriers listed above can be formulated using conventional methods that are known. These pharmaceutical compositions can be administered to a subject in a suited dose. The administration can be oral or parenteral, e.g. intravenous, intraperitoneal, subcutaneous, intramuscular, local, intranasal, intrabronchial or intradermal or via a catheter inserted at a site in an artery. The kind of dosage is determined by the attending physician according to the clinical factors. The person skilled in the art knows that the kind of dosage depends on different factors, such as body height and weight, body surface, age, gender or the general health condition of the patient, however, it also depends on the composition to be administered in the particular case, the time and kind of administration and on other medicaments which are possibly administered at the same time. A typical dose can, for example, be within a range between 0.01 and 10000 μg, with doses below or above this exemplary range being possible, in particular in consideration of the factors mentioned above. In general, with regular administration of the pharmaceutical preparation, the dose should be in a range between 10 ng units and 10 mg units per day and/or application interval. If the composition is administered intravenously, the dose should be within a range between 1 ng units and 0.1 mg units per kilogram body weight/minute.

The pharmaceutical composition of the invention can be administered locally or systemically. Preparations for parenteral administration comprise sterile aqueous or non-aqueous solutions, suspensions and emulsions. Examples of non-aqueous solvents are propylene glycol, polyethylene glycol, plant oils, such as e.g. olive oil, and organic ester compounds, such as e.g. ethyolate, which are suited for injection. Aqueous carriers comprise water, alcoholic/aqueous solutions, emulsions, suspensions, saline solutions and buffered media. Parenteral carriers include sodium chloride solutions, Ringer's dextrose, dextrose and sodium chloride, lactated Ringer's lactate and fixed oils. Intravenous carriers include e.g. fluid, nutrient and electrolyte replenishers (such as e.g. based on Ringer's dextrose). The pharmaceutical composition can also include preservatives and other additives, such as e.g. antimicrobial compounds, anti-oxidants, chelating agents and inert gases. Furthermore, depending on the intended use, compounds such as e.g. interleukins, growth factors, differentiation factors, interferons, chemotaxis proteins or an unspecific agent may be present.

Furthermore, the individual sequences of the siRNA 8 can also be administered in combination simultaneously or sequentially as well as in identical or different concentrations in order to silence a plurality of genes or degrade mRNAs efficiently.

FIG. 3 illustrates an additional effect which can further support the toxic effect of the siRNA 8 of the invention as described above. In this case, one or more nucleotide sequences (such as

| | |
|---|---|
| AAA, | (SEQ ID NO: 8) |
| UUU, | (SEQ ID NO: 9) |
| GCCA, | (SEQ ID NO: 10) |
| UGGC, | (SEQ ID NO: 11) |
| GUCCUUCAA, | (SEQ ID NO: 12) |
| UGUGU, | (SEQ ID NO: 13) |
| AUUUG, | (SEQ ID NO: 14) |
| GUUUU, | (SEQ ID NO: 15) |
| AUUUU, | (SEQ ID NO: 16) |
| CUUUU, | (SEQ ID NO: 17) |
| UUUUU | (SEQ ID NO: 18) |
| or | |
| GUUUG | (SEQ ID NO: 19)) |

which are known to trigger stress reactions in cell 2 that cannot be attributed to the binding of the siRNA 8 to one or more mRNAs, are additionally introduced into a siRNA 12. After the siRNA 12 has been introduced into the cell 2, the nucleotide sequences of the siRNA 12 having this effect do not reduce the expression of genes and the degradation of mRNAs (cf. the mRNA 3-6 shown in FIG. 3 that are not degraded by these nucleotide sequences) but induce unspecific stress reactions in the cell which occur in addition to the effect described in FIG. 2 and, thus, additionally contribute to the death of the cell 2.

### LIST OF REFERENCES USED

1, 8, 12—siRNA
2—cell
3, 4, 5, 6—gene-specific mRNA
7, 9, 10, 11—degraded gene-specific mRNA

1. Biologically active nucleotide molecules, comprising at least one nucleotide sequence targeting mRNA binding for selectively influencing cells, wherein said at least one nucleotide sequence of the nucleotide molecules is configured to bind to mRNA of a plurality of genes of the cells, thereby triggering a plurality of off-target effects which have a toxic effect on the cells by subjecting the cells to cell-killing stress.

2. The biologically active nucleotide molecules according to claim 1, comprising RNA, siRNA, PNA, DNA or LNA having a size of 10-300 bp.

3. The biologically active nucleotide molecules according to claim 1, wherein the nucleotide molecules further comprise sequences triggering, per se and without binding to an mRNA, stress reactions in the cells.

4. The biologically active nucleotide molecules according to 1, wherein the nucleotide molecules, to facilitate their introduction into the cells, are bound to cell-penetrating molecules or integrated in reagents.

5. The biologically active nucleotide molecules according to claim 1, wherein the nucleotide molecules contain at least one of the nucleotide sequences
GGUA, CGUC, CGUU, CCAA, AAGG, GGUG, CUCG,
CUCC, CUCU, CUUA, GGUC, GGUU, AAAG, AAAC, AAAU,
AAGA, AAGC, AAGU, AACA, AACG, AACC, AACU, AAUA,
CUUU, AAUG, AAUC, AAUU, AGGA, AGUG, AGUC, AGUU,
ACAA, ACAG, ACAC, ACAU, ACGA, ACGG, ACGC, ACGU,
ACCA, CAUU, CGAA, ACCG, ACCC, ACCU, ACUA, ACUG,
ACUC, ACUU, AUAA, GGAG, GGAC, GGAU, GGGA, GGGC,
GGGU, GGCA, GGCG, GGCC, GGCU, GCAA, GCAG, GCAC,
GCAU, AUAG, AUAC, AUAU, AUGA, AUGG, AUGC, AUGU,
AUCA, CGCG, CGCC, CGCU, AUCG, AUCC, AUCU, AUUA,
AUUG, AUUC, AUUU, GAAA, GAAG, GAAC, GAAU, GAGA,
GAGG, GAGC, GAGU, GACA, GACG, GACC, GACU, GAUA,
GAUG, GAUC, GAUU, GGAA, GCGA, GCGG, GCGC, GCGU,
GCCA, GCCG, GCCC, GCCU, GCUA, GCUG, GCUC, GCUU,
GUAA, GUAG, GUAC, GUAU, GUGA, GUGG, GUGC, GUGU,
GUCA, GUCG, GUCC, GUCU, GUUA, GUUG, GUUC, GUUU,
CAAA, CAAG, CAAC, CAAU, CAGA, CAGG, CAGC, CAGU,
CACA, CACG, CACC, CACU, CAUA, CAUG, CAUC, CGAG,
CGAC, CGAU, CGGA, CGGG, CGGC, CGGU, CGCA, CGUA,
CGUG, CCAG, CCAC, CCAU, CCGA, CCGG, CCGC, CCGU,
CCCA, AGAA, AGAG, AGAC, AGAU, CCCG, CCCU, AGGG,
AGGC, AGGU, AGCA, CCUA, CCUG, CCUC, CCUU, CUAA,
CUAG, CUAC, CUAU, AGCG, AGUA, CUGA, CUGG, CUGC,
CUGU, CUCA, CUUG, CUUC, AGCC, AGCU.

6. The biologically active molecules according to claim 1, selected from the group consisting of

| | |
|---|---|
| GUCUAUCAGCACAAUtt, | (SEQ ID NO: 1) |
| GCUUAACUGUAUCUGGAGCtt, | (SEQ ID NO: 2) |
| UUAACUGUAUCUGGAGCtt, | (SEQ ID NO: 3) |
| AACUGUAUCUGGAGCtt, | (SEQ ID NO: 4) |
| GCUCACCAAUGGAGAtt, | (SEQ ID NO: 5) |
| GGCUGAACAAAGGAGAtt | (SEQ ID NO: 6) |
| and | |
| UGGCUGGCUGGCUGGCtt. | (SEQ ID NO: 7) |

7. Pharmaceutical composition, comprising biologically active nucleotide molecules according to claim 1.

8. Method of treatment or prophylaxis of tumour diseases or virus-induced diseases, comprising administering the nucleotide molecules of claim 1.

9. Method of selectively killing eukaryotic cells, comprising transfecting the cells with the nucleotide molecules according to claim 1.

10. Method of selectively killing virus-infected cells, comprising transfecting the cells with the nucleotide molecules of claim 1.

11. Method of selectively killing prokaryotic cells, comprising transfecting the cells with the nucleotide molecules of claim 1.

12. The method of claim 2, further comprising administering the biologically active nucleotide molecules in combination with protease inhibitors.

13. Application kit for administration to target cells of the biologically active nucleotide molecules according to claim 1, comprising:
at least one ampoule (ampoule A) which contains the biologically active molecule;
at least one further ampoule (ampoule B) containing a transfection system;
at least one further ampoule (ampoule C) containing further components for binding to the biologically active molecules or the transfection system;
dilution and reaction buffers for the contents of ampoules A, B;
one or more probes and syringes with cannulas for injecting a mixture of the ampoule contents into a medium containing the target cells; and
instructions for use of said kit.

14. The biologically active nucleotide molecules according to claim 2, wherein the sequences triggering, per se and without binding to an mRNA, stress reactions in the cells comprise
AAA, UUU, GCCA, UGGC, GUCCUUCAA, UGUGU
AUUUG, GUUUU, AUUUU, CUUUU, UUUUU or GUUUG.

15. Method of treatment or prophylaxis of tumour diseases or virus induced diseases, comprising administering the pharmaceutical composition of claim 7.

16. The method of claim 9, wherein the eukaryotic cells are animal, plant or fungal cells.

17. The application kit according to claim 13, wherein the transfection system comprises cell-penetrating peptides, nanoparticles, polyethylenimines or lipids.


SEQUENCE LISTING (19 sequences)

SEQ ID NO: 1 - DNA, 17 bp, Artificial Sequence
  Description of Artificial Sequence Synthetic oligonucleotide
  Description of Combined DNA/RNA Molecule Synthetic oligonucleotide
  modified_base (16)..(17): 2′-Deoxythymidine
gucuaucagcacaautt

SEQ ID NO: 2 - DNA, 21 bp, Artificial Sequence
  Description of Artificial Sequence Synthetic oligonucleotide
  Description of Combined DNA/RNA Molecule Synthetic oligonucleotide
  modified_base (20)..(21): 2′-Deoxythymidine
gcuuaacuguaucuggagctt

SEQ ID NO: 3 - DNA, 19 bp, Artificial Sequence
  Description of Artificial Sequence Synthetic oligonucleotide
  Description of Combined DNA/RNA Molecule Synthetic oligonucleotide
  modified_base (18)..(19): 2′-Deoxythymidine
uuaacuguaucuggagctt

SEQ ID NO: 4 - DNA, 17 bp, Artificial Sequence
  Description of Artificial Sequence Synthetic oligonucleotide
  Description of Combined DNA/RNA Molecule Synthetic oligonucleotide
  modified_base (16)..(17): 2′-Deoxythymidine
aacuguaucuggagctt

SEQ ID NO: 5 - DNA, 17 bp, Artificial Sequence
  Description of Artificial Sequence Synthetic oligonucleotide
  Description of Combined DNA/RNA Molecule Synthetic oligonucleotide
  modified_base (16)..(17): 2′-Deoxythymidine
gcucaccaauggagatt

SEQ ID NO: 6 - DNA, 18 bp, Artificial Sequence
  Description of Artificial Sequence Synthetic oligonucleotide
  Description of Combined DNA/RNA Molecule Synthetic oligonucleotide
  modified_base (17)..(18): 2′-Deoxythymidine
ggcugaacaaaggagatt

SEQ ID NO: 7 - DNA, 18 bp, Artificial Sequence
  Description of Artificial Sequence Synthetic oligonucleotide
  Description of Combined DNA/RNA Molecule Synthetic oligonucleotide
  modified_base (17)..(18): 2′-Deoxythymidine
uggcuggcuggcuggctt

SEQ ID NO: 8 - RNA, 3 nt, Artificial Sequence
  Description of Artificial Sequence Synthetic oligonucleotide
aaa

SEQ ID NO: 9 - RNA, 3 nt, Artificial Sequence
  Description of Artificial Sequence Synthetic oligonucleotide
uuu

SEQ ID NO: 10 - RNA, 4 nt, Artificial Sequence
  Description of Artificial Sequence Synthetic oligonucleotide
gcca

SEQ ID NO: 11 - RNA, 4 nt, Artificial Sequence
  Description of Artificial Sequence Synthetic oligonucleotide
uggc

SEQ ID NO: 12 - RNA, 9 nt, Artificial Sequence
  Description of Artificial Sequence Synthetic oligonucleotide
guccuucaa

SEQ ID NO: 13 - RNA, 5 nt, Artificial Sequence
  Description of Artificial Sequence Synthetic oligonucleotide
ugugu

SEQ ID NO: 14 - RNA, 5 nt, Artificial Sequence
  Description of Artificial Sequence Synthetic oligonucleotide
auuug

SEQ ID NO: 15 - RNA, 5 nt, Artificial Sequence
  Description of Artificial Sequence Synthetic oligonucleotide
guuuu

SEQ ID NO: 16 - RNA, 5 nt, Artificial Sequence
  Description of Artificial Sequence Synthetic oligonucleotide
auuuu

SEQ ID NO: 17 - RNA, 5 nt, Artificial Sequence
  Description of Artificial Sequence Synthetic oligonucleotide
cuuuu

SEQ ID NO: 18 - RNA, 5 nt, Artificial Sequence
  Description of Artificial Sequence Synthetic oligonucleotide
uuuuu

SEQ ID NO: 19 - RNA, 5 nt, Artificial Sequence
  Description of Artificial Sequence Synthetic oligonucleotide
guuug